SEQUENCE LISTING (66 sequences)

SEQ ID NO: 1 - DNA, 40 bp, Artificial Sequence
  Modified sequence with linker; MI-2021-mVEGFA V1-5 ALX546 /5ThioMC6-D/ modified to be connected to /3AlexF546/
ttttttttttgcagctctgtctttctttggtctgcgctgc

SEQ ID NO: 2 - DNA, 40 bp, Artificial Sequence; identical to SEQ ID NO: 1
  MI-2021-mVEGFA V1-5

SEQ ID NO: 3 - DNA, 20 bp, Artificial Sequence
  MI-2021-mVEGFA V1-5
tctgtctttctttggtctgc

SEQ ID NO: 4 - DNA, 40 bp, Artificial Sequence
  Modified sequence with linker; MI-2021-mVEGF- 164 ALX647 /5ThioMC6-D/ modified to be connected to /3AlexF647/
ttttttttttccggtaaggctcacagtgattttctaccgg

SEQ ID NO: 5 - DNA, 40 bp, Artificial Sequence; identical to SEQ ID NO: 4
  MI-2021-mVEGF-164

SEQ ID NO: 6 - DNA, 22 bp, Artificial Sequence
  MI-2021-mVEGF-164
taaggctcacagtgattttcta

SEQ ID NO: 7 - DNA, 44 bp, Artificial Sequence
  Modified sequence with linker; MI_mENG 1 5ThioMC6-D/ modified to be connected to /3-Alx488
ttttttttttgcagctgcaactcagttccatcattacgggctgc

SEQ ID NO: 8 - DNA, 44 bp, Artificial Sequence; identical to SEQ ID NO: 7
  MI_mENG 1

SEQ ID NO: 9 - DNA, 24 bp, Artificial Sequence
  MI_mENG 1
tgcaactcagttccatcattacgg

SEQ ID NO: 10 - DNA, 24 bp, Artificial Sequence
  MI_mENG 1 comp
ccgtaatgatggaactgagttgca

SEQ ID NO: 11 - DNA, 40 bp, Artificial Sequence
  Modified sequence with linker; MI_mENG 2 5ThioMC6-D/ modified to be connected to /3-Alx488
ttttttttttgcagcactgtgatgttgactcttggcgctg

SEQ ID NO: 12 - DNA, 41 bp, Artificial Sequence
  MI_mENG 2
ttttttttttgcagcactgtgatgttgactcttggcgctgc

SEQ ID NO: 13 - DNA, 24 bp, Artificial Sequence
  MI_mENG 2
agcactgtgatgttgactcttggc

SEQ ID NO: 14 - DNA, 24 bp, Artificial Sequence
  MI_mENG 2 comp
gccaagagtcaacatcacagtgct

SEQ ID NO: 15 - DNA, 44 bp, Artificial Sequence
  Modified sequence with linker; MI_mENG 3 5ThioMC6-D/ modified to be linked to /3-Alx488
ttttttttttgctcgtttgaccttgcttcctggaaagatcgagc

SEQ ID NO: 16 - DNA, 44 bp, Artificial Sequence; identical to SEQ ID NO: 15
  MI_mENG 3

SEQ ID NO: 17 - DNA, 24 bp, Artificial Sequence
  MI_mENG 3
tttgaccttgcttcctggaaagat

SEQ ID NO: 18 - DNA, 24 bp, Artificial Sequence
  MI_mENG 3 comp
atctttccaggaagcaaggtcaaa

SEQ ID NO: 19 - DNA, 40 bp, Artificial Sequence
  Human sequence; MI_hENG 1 5ThioMC6-D/ modified with linker to be connected to /3Alx488
ttttttttttcgagcgagaagtggacacagggacggctcg

SEQ ID NO: 20 - DNA, 40 bp, Artificial Sequence; identical to SEQ ID NO: 19
  MI_hENG 1

SEQ ID NO: 21 - DNA, 20 bp, Artificial Sequence
  MI_hENG 1
gagaagtggacacagggacg

SEQ ID NO: 22 - DNA, 20 bp, Artificial Sequence
  MI_hENG 1 comp
cgtccctgtgtccacttctc

SEQ ID NO: 23 - DNA, 43 bp, Artificial Sequence
  Modified sequence with linker; MI-mHIF-1a_1 5ThioMC6-D modified to be connected to /3-Alx488
ttttttttttccggtattgtccttcgtctctgtttttgaccgg

SEQ ID NO: 24 - DNA, 43 bp, Artificial Sequence; identical to SEQ ID NO: 23
  MI_mHIF-1a_1

SEQ ID NO: 25 - DNA, 25 bp, Artificial Sequence
  MI_mHIF-1a_1
tattgtccttcgtctctgtttttga

SEQ ID NO: 26 - DNA, 25 bp, Artificial Sequence
  MI_mHIF-1a_1 comp
tcaaaaacagagacgaaggacaata

SEQ ID NO: 27 - DNA, 41 bp, Artificial Sequence
  Modified sequence with linker; MI-mHIF-1a_2 5ThioMC6-D/ modified to be connected to /3-Alx488
ttttttttttgcaccgtaaagaaacatcaggtaataggtgc

SEQ ID NO: 28 - DNA, 41 bp, Artificial Sequence; identical to SEQ ID NO: 27
  MI-mHIF-1a_2

SEQ ID NO: 29 - DNA, 21 bp, Artificial Sequence
  MI-mHIF-1a_2
gtaaagaaacatcaggtaata

SEQ ID NO: 30 - DNA, 21 bp, Artificial Sequence
  MI_mHIF-1a_2 comp
tattacctgatgtttctttac

SEQ ID NO: 31 - DNA, 45 bp, Artificial Sequence
  Modified sequence with linker; MI-mHIF-1a_3 5ThioMC6-D/ modified to be connected to /3-Alx488
ttttttttttcgagcattaaaagaacatattaaaaagagcgctcg

SEQ ID NO: 32 - DNA, 45 bp, Artificial Sequence; identical to SEQ ID NO: 31
  MI-mHIF-1a_3

SEQ ID NO: 33 - DNA, 25 bp, Artificial Sequence
  MI-mHIF-1a_3
attaaaagaacatattaaaaagagc

SEQ ID NO: 34 - DNA, 25 bp, Artificial Sequence
  MI_mHIF-1a_3 comp
gctctttttaatatgttcttttaat

SEQ ID NO: 35 - DNA, 44 bp, Artificial Sequence
  Modified sequence with linker; MI_2 Scr 5ThioMC6-D/ modified to be connected to /3-Alx488
ttttttttttgcagcataactcgtccgtccgtaccgaccgctgc

SEQ ID NO: 36 - DNA, 44 bp, Artificial Sequence; identical to SEQ ID NO: 35
  MI_2 Scr

SEQ ID NO: 37 - DNA, 24 bp, Artificial Sequence
  MI_2 Scr
ataactcgtccgtccgtaccgacc

SEQ ID NO: 38 - DNA, 24 bp, Artificial Sequence
  MI-2 Scr Comp
ggtcggtacggacggacgagttat

SEQ ID NO: 39 - RNA, 33 nt, Artificial Sequence
  All nucleotides are 2′-MeO protected; MI-01- 2022-mHIF-1 2326; 5′-/5ThioMC6-D/ modified to be connected to /-3′
ccgguauuguccuucgucucuguuuuugaccgg

SEQ ID NO: 40 - RNA, 33 nt, Artificial Sequence; identical to SEQ ID NO: 39
  All nucleotides are 2′-MeO protected

SEQ ID NO: 41 - RNA, 25 nt, Artificial Sequence
  All nucleotides are 2′-MeO protected
uauuguccuucgucucuguuuuuga

SEQ ID NO: 42 - RNA, 33 nt, Artificial Sequence
  All nucleotides are 2′-MeO protected; MI-01- 2022-mHIF-1 Neg 2326; 5′-/5ThioMC6-D/ modified to be connected to /-3′
ccgguuuaguuccuguucuguugucuucaccgg

SEQ ID NO: 43 - RNA, 33 nt, Artificial Sequence; identical to SEQ ID NO: 42
  All nucleotides are 2′-MeO protected; MI-01- 2022-mHIF-1 Neg

SEQ ID NO: 44 - RNA, 25 nt, Artificial Sequence
  All nucleotides are 2′-MeO protected; MI-01- 2022-mHIF-1 Neg
uuuaguuccuguucuguugucuuca

SEQ ID NO: 45 - RNA, 31 nt, Artificial Sequence
  All nucleotides are 2′-MeO protected; MI-01- 2022-mHIF-1 4661; 5′-/5ThioMC6-D/ modified to be connected to /-3′
gcaccguaaagaaacaucagguaauaggugc

SEQ ID NO: 46 - RNA, 31 nt, Artificial Sequence; identical to SEQ ID NO: 45
  All nucleotides are 2′-MeO protected; MI-01- 2022-mHIF-1

SEQ ID NO: 47 - RNA, 21 nt, Artificial Sequence
  All nucleotides are 2′-MeO protected; MI-01- 2022-mHIF-1
guaaagaaacaucagguaaua

SEQ ID NO: 48 - RNA, 31 nt, Artificial Sequence
  All nucleotides are 2′-MeO protected; MI-01- 2022-mHIF-1 Neg 4661; 5′-/5ThioMC6-D/ modified to be connected to /-3′
gcaccauagagacaauauuagaagacggugc

SEQ ID NO: 49 - RNA, 31 nt, Artificial Sequence; identical to SEQ ID NO: 48
  All nucleotides are 2′-MeO protected; MI-01- 2022-mHIF-1 Neg

SEQ ID NO: 50 - RNA, 21 nt, Artificial Sequence
  All nucleotides are 2′-MeO protected; MI-01- 2022-mHIF-1 Neg
auagagacaauauuagaagac

SEQ ID NO: 51 - RNA, 35 nt, Artificial Sequence
  All nucleotides are 2′-MeO protected; MI-01- 2022-mHIF-1; 5′-/5ThioMC6-D/ modified to be connected to /-3′
cgagcauuaaaagaacauauuaaaaagagcgcucg

SEQ ID NO: 52 - RNA, 35 nt, Artificial Sequence; identical to SEQ ID NO: 51
  All nucleotides are 2′-MeO protected; MI-01- 2022-mHIF-1

SEQ ID NO: 53 - RNA, 25 nt, Artificial Sequence
  All nucleotides are 2′-MeO protected; MI-01- 2022-mHIF-1
auuaaaagaacauauuaaaaagagc

SEQ ID NO: 54 - RNA, 33 nt, Artificial Sequence
  All nucleotides are 2′-MeO protected; MI-01- 2022-mENG seq-1; 5ThioMC6-D/ modified to be connected to /-3′
gcagcugcaacucaguuccaucauuacgggcug

SEQ ID NO: 55 - RNA, 34 nt, Artificial Sequence
  All nucleotides are 2′-MeO protected; MI-01- 2022-mENG seq-1
gcagcugcaacucaguuccaucauuacgggcugc

SEQ ID NO: 56 - RNA, 24 nt, Artificial Sequence
  All nucleotides are 2′-MeO protected; MI-01- 2022-mENG seq-1
ugcaacucaguuccaucauuacgg

SEQ ID NO: 57 - RNA, 31 nt, Artificial Sequence
  All nucleotides are 2′-MeO protected; MI-01- 2022-mENG seq-2; 5′-/5ThioMC6-D/ modified to be connected to /-3′
gcagcacugugauguugacucuuggcgcugc

SEQ ID NO: 58 - RNA, 31 nt, Artificial Sequence; identical to SEQ ID NO: 57
  All nucleotides are 2′-MeO protected; MI-01- 2022-mENG seq-2

SEQ ID NO: 59 - RNA, 21 nt, Artificial Sequence
  All nucleotides are 2′-MeO protected; MI-01- 2022-mENG seq-2
acugugauguugacucuuggc

SEQ ID NO: 60 - RNA, 34 nt, Artificial Sequence
  All nucleotides are 2′-MeO protected; MI-01- 2022-mENG seq-3; 5′-/5ThioMC6-D/ modified to be connected to /-3′
gcucguuugaccuugcuuccuggaaagaucgagc

SEQ ID NO: 61 - RNA, 34 nt, Artificial Sequence; identical to SEQ ID NO: 60
  All nucleotides are 2′-MeO protected; MI-01- 2022-mENG seq-3

SEQ ID NO: 62 - RNA, 24 nt, Artificial Sequence
  All nucleotides are 2′-MeO protected; MI-01- 2022-mENG seq-3
uuugaccuugcuuccuggaaagau

SEQ ID NO: 63 - DNA, 24 bp, Artificial Sequence
  All nucleotides are 2′-MeO protected; For ENG seq-1 Mus musculus endoglin (Eng), transcript variant 1, mRNA NM_007932.2
gccaagagtcaacatcacagtgct

SEQ ID NO: 64 - DNA, 24 bp, Artificial Sequence
  All nucleotides are 2′-MeO protected; For ENG seq-2 Mus musculus endoglin (Eng), transcript variant 1, mRNA NM_007932.2
ccgtaatgatggaactgagttgca

SEQ ID NO: 65 - DNA, 24 bp, Artificial Sequence
  All nucleotides are 2′-MeO protected; For ENG seq-3 Mus musculus endoglin (Eng), transcript variant 1, mRNA NM_007932.2
atctttccaggaagcaaggtcaaa

SEQ ID NO: 66 - DNA, 24 bp, Artificial Sequence
  Mouse VCAM1
gcctccaccagactgtacgatcct

(19) United States
(12) Patent Application Publication
(10) Pub. No.: US 2022/0233714 A1
(43) Pub. Date: Jul. 28, 2022

(54) NANOGOLD-DNA BIOCONJUGATES AND METHODS OF USE THEREOF

(71) Applicant: Vanderbilt University, Nashville, TN (US)

(72) Inventors: MD Imam Uddin, Nashville, TN (US); John S. Penn, Brentwood, TN (US); Gary W. McCollum, Joelton, TN (US)

(21) Appl. No.: 17/586,560

(22) Filed: Jan. 27, 2022

Related U.S. Application Data
(60) Provisional application No. 63/142,011, filed on Jan. 27, 2021.

(51) Int. Cl. A61K 47/69; C12N 15/113; A61K 31/712; A61K 31/7125

(52) U.S. Cl. CPC A61K 47/6929; C12N 15/113; A61K 31/712; B82Y 5/00; C12N 2310/321; C12N 2310/531; C12N 2310/11; A61K 31/7125

(57) ABSTRACT

The present disclosure relates to nanoparticles and methods for detecting nucleic acids and methods of inhibiting mRNA expression.

### CROSS REFERENCE TO RELATED APPLICATIONS

This application claims the benefit of U.S. Provisional Patent Application Ser. No. 63/142,011, filed Jan. 27, 2021, the disclosure of which is expressly incorporated herein by reference.

### STATEMENT REGARDING FEDERALLY SPONSORED RESEARCH

This invention was made with government support under Grant No. R01 EY023397 and R01 EY029693 awarded by the National Institutes of Health. The government has certain rights in the invention.

### STATEMENT REGARDING SEQUENCE LISTING

Applicant submits herewith a Sequence Listing in computer readable form and in compliance with 37 C.F.R. §§ 1.821-1.825. This sequence listing is in ASCII TXT format with filename “10644-123US1_2022_01_27 Sequence,” a 15,220 byte file size, and creation date of Jan. 27, 2022. The content of the Sequence Listing is hereby incorporated by reference.

### FIELD

The present disclosure relates to nanoparticles and methods for detecting nucleic acids and methods of inhibiting mRNA expression.

### BACKGROUND

A major challenge for in vivo molecular imaging and inhibition of mRNAs in living systems is that unmodified oligonucleotides are unstable and exhibit rapid renal clearance from circulation, leading to minimal bioavailability in target tissues. What is needed are novel compounds, compositions, and methods for detecting RNAs and methods of inhibiting mRNA expression.

The compounds, compositions, and methods disclosed herein address these and other needs.

### SUMMARY

Disclosed herein are nanoparticles and methods for detecting nucleic acids and methods of inhibiting mRNA expression. The inventors have developed nanogold DNA bioconjugates that provide delivery of nucleic acids into cells, for use in methods of imaging and inhibiting RNAs, without the use of toxic transfection agents.

In some aspects, disclosed herein is a nanoparticle, comprising:
a short hairpin DNA (shDNA), wherein the shDNA comprises an anti-sense oligonucleotide complementary to a target sequence of an endoglin mRNA; and
a colloidal gold nanoparticle conjugated to the shDNA.

In some embodiments, the nanoparticle further comprises a fluorescent dye conjugated to the shDNA.

In some embodiments, the shDNA comprises SEQ ID NO:21.

In some embodiments, the colloidal gold nanoparticle comprises an additional anti-sense oligonucleotide complementary to a second target sequence.

In some embodiments, the additional anti-sense oligonucleotide comprises a sequence complementary to a target sequence of a VCAM-1 or HIF-1α mRNA.

In some embodiments, the shDNA comprises about 15-45 nucleotides. In some embodiments, the target sequence of the endoglin mRNA is about 21 nucleotides.

In some embodiments, the shDNA comprises at least one chemically modified nucleotide. In some embodiments, the at least one chemically modified nucleotide comprises 2′-O-methyl (2′MeO).

In some embodiments, the colloidal gold nanoparticle is about 15 nm in diameter. In some embodiments, the colloidal gold nanoparticle is about 1.4 nm in diameter.

In some embodiments, the nanoparticle is conjugated to the shDNA by a linker. In some embodiments, the linker comprises a C-6 hexane linker.

In some embodiments, the fluorescent dye is cyanine-3 (Cy3).

In some aspects, disclosed herein is a method for inhibiting the expression of an RNA, comprising:

introducing a nanoparticle into a cell or a tissue, the nanoparticle comprising:
a short hairpin DNA sequence (shDNA), wherein the shDNA sequence comprises an anti-sense oligonucleotide complementary to a target sequence of an RNA; and
a colloidal gold nanoparticle conjugated to the shDNA;
allowing the anti-sense oligonucleotide to bind the target sequence; and
wherein the binding of the anti-sense oligonucleotide to the target sequence inhibits the expression of the RNA.

In some embodiments, the cell or tissue is an ocular cell or tissue.

In some embodiments, the RNA is selected from an endoglin mRNA, a VCAM-1 mRNA, a HIF-1α mRNA, or a VEGF mRNA.

In some embodiments, the RNA is an endoglin mRNA. In some embodiments, the RNA is a VCAM-1 mRNA.

In some aspects, disclosed herein is a method for treating a retinal disease in a subject, comprising:
administering a therapeutically effective amount of a nanoparticle to the subject, wherein the nanoparticle comprises:
   a short hairpin DNA sequence (shDNA), wherein the shDNA sequence comprises an anti-sense oligonucleotide complementary to a target sequence of an RNA; and
   a colloidal gold nanoparticle conjugated to the shDNA.

In some embodiments, the retinal disease is selected from age-related macular degeneration (AMD), retinopathy of prematurity (ROP), diabetic retinopathy (DR), or branch retinal vein occlusion (BRVO).

In some aspects, disclosed herein is a method for detecting an RNA, comprising:
introducing a nanoparticle into a cell or a tissue, the nanoparticle comprising:
   a short hairpin DNA sequence (shDNA), wherein the shDNA sequence comprises an anti-sense oligonucleotide complementary to a target sequence of an RNA;
   a colloidal gold nanoparticle conjugated to the shDNA; and
   a fluorescent dye conjugated to the shDNA;
allowing the anti-sense oligonucleotide to bind the target sequence; and
detecting the fluorescent dye after the anti-sense oligonucleotide binds to the target sequence.

In some aspects, disclosed herein is a nanoparticle, comprising:
a short hairpin DNA (shDNA), wherein the shDNA comprises an anti-sense oligonucleotide complementary to a target sequence of an endoglin, HIF-1α, or VEGF mRNA, or a combination thereof; and a colloidal gold nanoparticle conjugated to the shDNA.

### BRIEF DESCRIPTION OF THE DRAWINGS

The accompanying figures, which are incorporated in and constitute a part of this specification, illustrate several aspects described below.

FIGS. 1A-1D. Schematic drawing, hybridization motif, specificity and sensitivity of hAuNP. Two different types of nano-gold bioconjugates were designed and synthesized. (FIG. 1A) In probe type-1, 15 nm spherical gold nanoparticles were designed and functionalized with single or multiple targeted hairpin-DNA/RNA oligonucleotides incorporating anti-sense sequence specific for mouse Endoglin (ENG) mRNA, or VEGFA mRNA, or VCAM-1 mRNA, or HIF-1alpha mRNA (AS-hAuNP) allowing multi-targeted therapy, or a scrambled version of this sequence (NS-hAuNP). (FIG. 1B) In probe type-2, 1.4 nm gold nanoparticles were functionalized with a single hairpin-DNA/RNA oligonucleotide incorporating anti-sense sequence specific for mouse ENG mRNA, or VEGFA mRNA, or VCAM-1 mRNA, or HIF-1alpha mRNA (AS-hAuNP) or a scrambled version of this sequence (NS-hAuNP). The DNA hairpin-loops are modified on their 5′ ends with a thiol (SH) group and coupled to a maleimide group, which is connected to the gold surface through a phosphine-gold (Au—P) bond. The dye is linked to the 3′ end of the oligonucleotide through an O—(CH2)7-amide linkage and is quenched. Hybridization of the target mRNA to the anti-sense recognition sequence causes the hairpin to open increasing the distance between the fluorophore and the gold surface, resulting in fluorescence emission. (FIG. 1C) The AS-mENG hAuNP is highly specific for its complementary sequence. The hybridization kinetics of the AS-mENG hAuNP in the presence of the complementary target sequence and non-sense (NS) sequence confirmed the specificity of the AS-hAuNP for the exogenous mENG oligo compared to the scrambled sequence (NS-oligo). (FIG. 1D) Transmission electron microscopy (TEM) imaging of AS-hAuNP shows the monodispersity of the nanoparticles in highly ionic media (PBS).

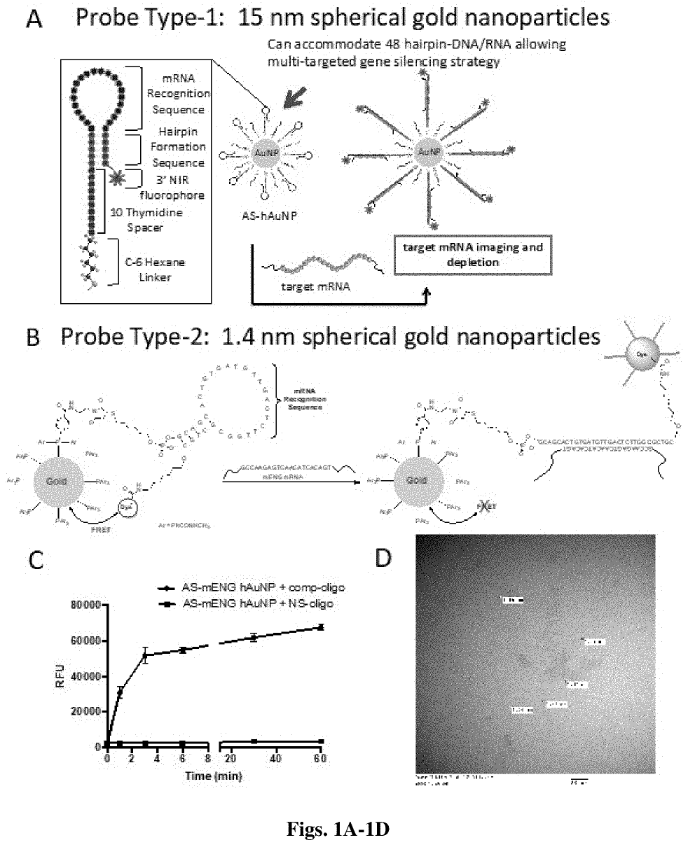

FIGS. 2A-2M. Fluorescence and confocal imaging of MRMECs treated with AS-hAuNP and NS-hAuNP in complete growth medium. Cells were cultured on microscope slides and treated under hypoxia to induce endoglin mRNA or normoxic condition. After a 24 hour incubation, the media were aspirated and fresh medium was added to each culture. The cells were imaged using fluorescence and confocal microscopy. (FIGS. 2A, 2B) Strong fluorescence emission was only detected in cells treated with AS-hAuNP under hypoxic condition. (FIGS. 2C, 2D) Only minimal fluorescence was detected when NS-hAuNP was used under both hypoxic and normoxic conditions. (FIGS. 2E through 2L) Confocal imaging of the MRMECs treated with AS-hAuNP under hypoxia reveled that the fluorescence is localized in cytosol and perinuclearly suggesting that the probe retains in the cytosol. (FIG. 2M) Fluorescence from AS-hAuNP (Green) showing its sensitivity and specificity.

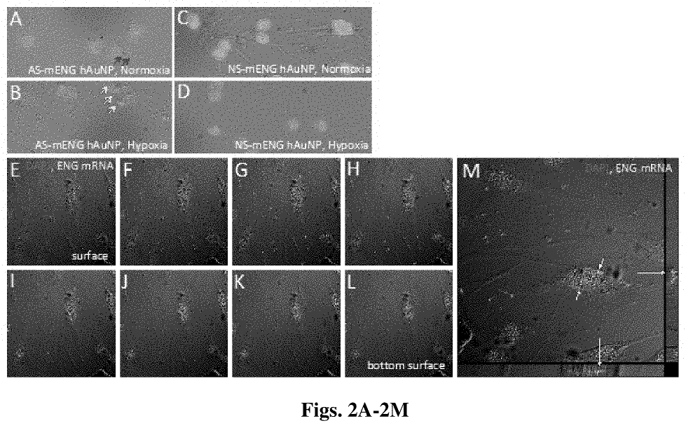

FIG. 3 Gold nanoparticles were functionalized with hairpin-DNA oligonucleotide incorporating anti-sense sequence specific for target mRNA (AS-hAuNP) or a scrambled version of this sequence (NS-hAuNP). Target mRNA depletion was achieved by more than 67% using AS-hAuNP as measured by qRT-PCR, without using any transfection reagents.

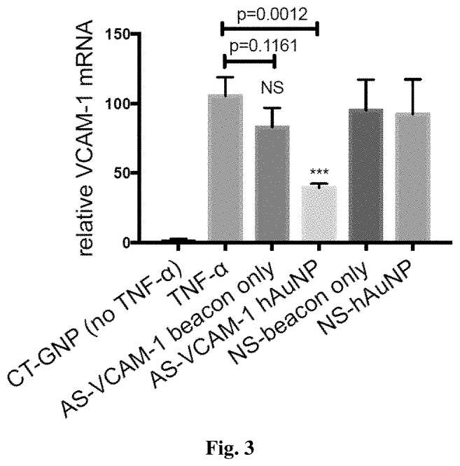

FIG. 4 Inhibition of target mRNA using hAuNP is highly effective therapy for age-related macular degeneration (AMD). In this figure, 7/10 and 5/11 reflects day of single intravitreal injection followed by day of collecting choroidal tissues for analysis. Mouse specific anti-VEGF neutralizing antibody was used as therapeutic treatment control group.

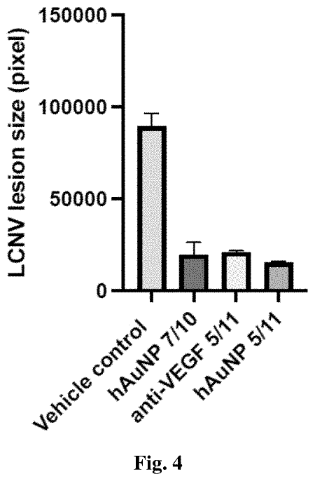

### DETAILED DESCRIPTION

Disclosed herein are nanoparticles and methods for detecting nucleic acids and methods of inhibiting mRNA expression. The inventors have developed nanogold DNA bioconjugates that provide delivery of the oligonucleotides into cells for methods of imaging and inhibiting RNAs, without the use of toxic transfection agents. Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

The following definitions are provided for the full understanding of terms used in this specification.

### Terminology

As used herein, the article “a,” “an,” and “the” means “at least one,” unless the context in which the article is used clearly indicates otherwise.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed herein.

The term “nucleic acid” as used herein means a polymer composed of nucleotides, e.g., deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin; for example, promoters derived from viruses or from other organisms can be used in the compositions, systems, or methods described herein. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cells and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol I promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R—U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It is appreciated by those skilled in the art that the design of the expression vector can depend on such factors as the choice of the host cell to be transformed, the level of expression desired, etc.

The term “recombinant” refers to a human manipulated nucleic acid (e.g., polynucleotide) or a copy or complement of a human manipulated nucleic acid (e.g., polynucleotide), or if in reference to a protein (i.e, a “recombinant protein”), a protein encoded by a recombinant nucleic acid (e.g., polynucleotide). In embodiments, a recombinant expression cassette comprising a promoter operably linked to a second nucleic acid (e.g., polynucleotide) may include a promoter that is heterologous to the second nucleic acid (e.g., polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g., polynucleotides) combined in such a way that the nucleic acids (e.g., polynucleotides) are extremely unlikely to be found in nature. For instance, human-manipulated restriction sites or plasmid vector sequences may flank or separate the promoter from the second nucleic acid (e.g., polynucleotide). One of skill will recognize that nucleic acids (e.g., polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g., polynucleotide) may include a promoter that is heterologous to the second nucleic acid (e.g., polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g., polynucleotide) may include a terminator that is heterologous to the second nucleic acid (e.g., polynucleotide) as the result of human manipulation. In some embodiments, the expression cassette comprises a promoter operably linked to a second nucleic acid (e.g., polynucleotide) and a terminator operably linked to the second nucleic acid (e.g., polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotides or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g., enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g., modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

The term “about” as used herein when referring to a measurable value such as an amount, a percentage, and the like, is meant to encompass variations of ±20%, ±10%, ±5%, or ±1% from the measurable value.

### Nanoparticles and Methods

Disclosed herein is the design and synthesis of a series of oligonucleotide conjugated nano-gold colloids for applications in silencing and imaging specific disease mRNA biomarkers in target tissues without any added transfection reagents. In addition, these nano-probes have been used for imaging mRNA in live retinal cells. These nanoprobes can overcome the limitations of current siRNA based gene silencing strategies that requires the use of transfection reagents. These added transfection reagents are toxic to the primary cells and tissues. The nano-probes can be delivered to the neovascular lesions of laser-induced choroidal neovascularization (LCNV) and in neovascular lesions of oxygen-induced retinopathy and could be used for imaging mRNA biomarkers in tissues. These new probes are not acutely toxic to the retinal cells and tissues.

These non-toxic nanogold gene silencing agents can overcome the limitations of transfection and delivery of siRNA methods and are used as an imaging tool for the detection and depletion of faulty genes in vascular diseases including age-related macular degeneration (AMD), retinopathy of prematurity (ROP), or diabetic retinopathy (DR). Gene silencing strategies for the treatment of ocular NV using siRNAs has been limited by low specific delivery to the target tissues and off-target effects. These new gold nanoprobes can overcome these limitations.

These nanogold gene silencing agents also possess stronger target mRNA hybridization kinetics and stability properties than current therapeutics. When present at a target site, hAuNP binds and sequesters target messenger mRNA to suppress translation of proteins involved in retinal disease pathology. The inventors have found that inflammatory proteins induced by laser-induced choroidal neovascularization (LCNV) are suppressed upon treatment with hAUNPs.

Clinical and preclinical applications include, for example, diagnostic and therapeutic applications in detection of an mRNA biomarker in age-related macular degeneration (AMD), retinopathy of prematurity (ROP), diabetic retinopathy (DR), and/or branch retinal vein occlusion (BRVO).

In some aspects, disclosed herein is a nanoparticle, comprising:
a short hairpin DNA (shDNA), wherein the shDNA comprises an anti-sense oligonucleotide complementary to a target sequence of an endoglin mRNA; and
a colloidal gold nanoparticle conjugated to the shDNA.

In some embodiments, the nanoparticle further comprising a fluorescent dye conjugated to the shDNA.

In some embodiments, the shDNA comprises SEQ ID NO:21.

In some embodiments, the colloidal gold nanoparticle comprises an additional anti-sense oligonucleotide complementary to a second target sequence.

In some embodiments, the additional anti-sense oligonucleotide comprises a sequence complementary to a target sequence of a VCAM-1 or HIF-1α mRNA.

In some embodiments, the shDNA comprises about 15-45 nucleotides. In some embodiments, the target sequence of the endoglin mRNA is about 21 nucleotides.

In some embodiments, the shDNA comprises at least one chemically modified nucleotide. In some embodiments, the at least one chemically modified nucleotide comprises 2′-O-methyl (2′MeO).

In some embodiments, the colloidal gold nanoparticle is about 15 nm in diameter. In some embodiments, the colloidal gold nanoparticle is about 1.4 nm in diameter. In some embodiments, a nanoparticle can range from about 0.1 nm to about 1000 nm, from about 1 nm and about 500 nm, from about 5 nm and about 100 nm, from about 10 nm and about 50 nm, or from about 15 nm and about 30 nm. In some embodiments, the colloidal gold nanoparticle is about 1 nm, 1.4 nm, 1.5 nm, 3 nm, 5 nm, 10 nm, 15 nm, 20 nm, 25 nm, 30 nm, 40 nm, 50 nm, 100 nm, 500 nm, or more, in diameter.

In some embodiments, the nanoparticle is conjugated to the shDNA by a linker. In some embodiments, the linker comprises a C-6 hexane linker.

In some embodiments, the fluorescent dye is cyanine-3 (Cy3). In some embodiments, the fluorescent dye is an Alexa Fluor dye. In some embodiments, the fluorescent dye is Alexa Fluor 546. In some embodiments, the fluorescent dye is Alexa Fluor 647. In some embodiments, the fluorescent dye is Alexa Fluor 488.

In some aspects, disclosed herein is a method for inhibiting the expression of an RNA, comprising:
introducing a nanoparticle into a cell or a tissue, the nanoparticle comprising:
   a short hairpin DNA sequence (shDNA), wherein the shDNA sequence comprises an anti-sense oligonucleotide complementary to a target sequence of an RNA; and
   a colloidal gold nanoparticle conjugated to the shDNA;
allowing the anti-sense oligonucleotide to bind the target sequence; and
wherein the binding of the anti-sense oligonucleotide to the target sequence inhibits the expression of the RNA.

In some embodiments, the cell or tissue is an ocular cell or tissue.

In some embodiments, the RNA is selected from an endoglin mRNA, a VCAM-1 mRNA, a HIF-1α mRNA, or a VEGF (VEGFA) mRNA.

In some embodiments, the RNA is an endoglin mRNA. In some embodiments, the RNA is a VCAM-1 mRNA.

In some aspects, disclosed herein is a method for treating a retinal disease in a subject, comprising:
administering a therapeutically effective amount of a nanoparticle to the subject, wherein the nanoparticle comprises:
   a short hairpin DNA sequence (shDNA), wherein the shDNA sequence comprises an anti-sense oligonucleotide complementary to a target sequence of an RNA; and
   a colloidal gold nanoparticle conjugated to the shDNA.

In some embodiments, the retinal disease is selected from age-related macular degeneration (AMID), retinopathy of prematurity (ROP), or diabetic retinopathy (DR). and branch retinal vein occlusion (BRVO).

In some aspects, disclosed herein is a method for detecting an RNA, comprising:
introducing a nanoparticle into a cell or a tissue, the nanoparticle comprising:
   a short hairpin DNA sequence (shDNA), wherein the shDNA sequence comprises an anti-sense oligonucleotide complementary to a target sequence of an RNA;
   a colloidal gold nanoparticle conjugated to the shDNA; and
   a fluorescent dye conjugated to the shDNA;
allowing the anti-sense oligonucleotide to bind the target sequence; and
detecting the fluorescent dye after the anti-sense oligonucleotide binds to the target sequence.

In some embodiments, the RNA is an mRNA. In some embodiments, the RNA comprises an endoglin mRNA. In some embodiments, the shDNA sequence comprises SEQ ID NO:9, SEQ ID NO:13, or SEQ ID NO:17. In some embodiments, the RNA comprises a human endoglin mRNA. In some embodiments, the shDNA sequence comprises SEQ ID NO:21.

In some embodiments, the RNA comprises a HIF-1α mRNA. In some embodiments, the shDNA sequence comprises SEQ ID NO:25, SEQ ID NO:29, or SEQ ID NO:33. In some embodiments, the RNA comprises a human HIF-1α mRNA.

In some embodiments, the shDNA sequence comprises SEQ ID NO:9, SEQ ID NO:13, SEQ ID NO:17, or SEQ ID NO:21, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO:9, SEQ ID NO:13, SEQ ID NO:17, or SEQ ID NO:21.

In some embodiments, the shDNA sequence comprises SEQ ID NO:25, SEQ ID NO:29, or SEQ ID NO:33, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO:25, SEQ ID NO:29, or SEQ ID NO:33.

In some embodiments, the RNA comprises a VEGF mRNA. In some embodiments, the shDNA sequence comprises SEQ ID NO:3 or SEQ ID NO:6. In some embodiments, the RNA comprises a human VEGF mRNA.

In some embodiments, the shDNA sequence comprises SEQ ID NO:3 or SEQ ID NO:6, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO:3 or SEQ ID NO:6.

In some embodiments, the shDNA sequence comprises SEQ ID NO:66, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO:66.

In some embodiments, the RNA comprises an endoglin mRNA, or a fragment thereof, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to the endoglin mRNA, or the fragment thereof. In some embodiments, the RNA comprises a HIF-1α mRNA, or a fragment thereof, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to the HIF-1α mRNA, or the fragment thereof. In some embodiments, the RNA comprises a VEGF mRNA, or a fragment thereof, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to the VEGF mRNA, or the fragment thereof. In some embodiments, the RNA comprises a VCAM1 mRNA, or a fragment thereof, or a sequence at least 60% (for example, at least 60%, at least 65%, at least 70%, at least 75%, at least 80%, at least 85%, at least 90%, at least 95%, at least 96%, at least 97%, at least 98%, at least 99%) identical to the VCAM1 mRNA, or the fragment thereof.

In some embodiments, the shDNA sequence comprises about 15-45 nucleotides. In some embodiments, the shDNA sequence comprises about 20-40 nucleotides. In some embodiments, the shDNA sequence comprises about 25-35 nucleotides. In some embodiments, the shDNA sequence comprises about 30-34 nucleotides. In some embodiments, the shDNA sequence comprises about 15, about 20, about 25, about 30, about 35, about 40, about 45, or more nucleotides.

In some embodiments, the antisense oligonucleotide is about 21 nucleotides. In some embodiments, the antisense oligonucleotide is about 10-35 nucleotides. In some embodiments, the antisense oligonucleotide is about 15-30 nucleotides. In some embodiments, the antisense oligonucleotide is about 18-25 nucleotides. In some embodiments, the antisense oligonucleotide is about 20-24 nucleotides. In some embodiments, the antisense oligonucleotide is about 15, 16, 17, 18, 19, 20, 21, 22, 23, 24, 25, 26, 27, 28, 29, 30, or more nucleotides.

In some embodiments, the shDNA comprises at least one chemically modified nucleotide. In some embodiments, the at least one chemically modified nucleotide comprises a chemically modified nucleobase, a chemically modified ribose, a chemically modified phosphodiester linkage, or a combination thereof.

In some embodiments, the at least one chemically modified nucleotide is a chemically modified ribose. In some embodiments, the chemically modified ribose is 2′-O-methyl (2′-O-Me or 2′MeO or 2′-MeO) or 2′-fluoro (2′-F). In some embodiments, the chemically modified ribose is 2′-O-methyl (2′MeO). In some embodiments, the chemically modified ribose is 2′-fluoro (2′-F).

In some embodiments, the at least one chemically modified nucleotide is a chemically modified phosphodiester linkage. In some embodiments, the chemically modified phosphodiester linkage is phosphorothioate (PS). In some embodiments, all the nucleotides comprise chemically modified phosphodiester linkages. In some embodiments, the chemically modified phosphodiester linkages are phosphorothioate (PS).

In some embodiments, the at least one chemically modified nucleotide is a locked nucleic acid (LNA). Locked nucleic acids (LNA) can be used to stabilize the probe for in vivo delivery.

In some embodiments, the fluorescent dye is cyanine-3 (Cy3). In some embodiments, the fluorescent dye is Cy5.

In some embodiments, the cell or tissue is an ocular cell or tissue.

In some embodiments, the detection of the fluorescent dye is compared to a control (for example, a control sample, or a control probe). In some embodiments, the increased fluorescence (as compared to a control) indicates detection of the nucleic acid (for example, an RNA).

In some embodiments, the nucleic acids herein are recombinant. In some embodiments, the nucleic acids herein are isolated. In some embodiments, the probes herein are recombinant. In some embodiments, the nanoparticle and/or oligonucleotides herein are isolated.

The nanoparticles herein are used for imaging mRNAs and for inhibiting expression of mRNAs, including but not limited to endoglin, HIF-1α, VCAM-1, or VEGF mRNA. While the shDNAs herein have targeted selected sequences, any other fragment sequence that can specifically bind the mRNA can also be used. The accession number for human endoglin (ENG) mRNA is: NM_001114753.2; and the accession number for human HIF-1alpha is NM_001243084.1. Accession numbers for all genes can be found at the National Center for Biotechnology Information website (ncbi.nlm.nih.gov). For human VCAM-1, the primary (citable) accession number is P19320 and VEGFA primary (citable) accession number is P15692. In some embodiments, the RNA comprises a HIF-1α, VCAM-1, or VEGF mRNA, or a fragment thereof, or a sequence at least 60% (e.g., at least 60%, at least 65%, at least 70%, at least 75%, at least 80%, at least 85%, at least 90%, at least 95%, at least 96%, at least 97%, at least 98%, at least 99%) identical to the HIF-1α, VCAM-1, and VEGF mRNA, or the fragment thereof.

In some aspects, instead of a short hairpin DNA, a short hairpin RNA sequence can be used. In some embodiments, the RNA sequences comprise SEQ ID NO:39-62, which show the hairpin RNA sequences and the antisense oligonucleotide sequences that can bind to the target RNA.

In some aspects, disclosed herein is a method for inhibiting the expression of an RNA, comprising:
introducing a nanoparticle into a cell or a tissue, the nanoparticle comprising:
   a short hairpin RNA sequence (shRNA), wherein the shRNA sequence comprises an anti-sense oligonucleotide complementary to a target sequence of an RNA; and
   a colloidal gold nanoparticle conjugated to the shRNA;
allowing the anti-sense oligonucleotide to bind the target sequence; and
wherein the binding of the anti-sense oligonucleotide to the target sequence inhibits the expression of the RNA.

In some embodiments, the RNA is an mRNA. In some embodiments, the cell or tissue is an ocular cell or tissue. In some embodiments, the cell or tissue is a retinal cell or tissue.

In some embodiments, the retinal disease is selected from proliferative diabetic retinopathy (PDR), age-related macular degeneration (AMD), retinopathy of prematurity (ROP), retinal vein occlusion, or ocular cancer. In some embodiments, the retinal disease is wet AMD. In some embodiments, the retinal disease is dry AMD.

In some embodiments, the nanoparticles disclosed herein can be administered in combination with an additional therapeutic agent. Ranibizumab can be used to treat macular edema caused by diabetic retinopathy (DR). Ranibizumab can also be used to treat choroidal neovascularization in AMD. Another drug, bevacizumab (trade name Avastin), can also be used to treat AMD. Laser therapy can be used to treat advanced ROP. Cryotherapy can be used to freeze a specific part of the eye that extends beyond the edges of the retina. Ranibizumab or bevacizumab can be used to treat retinal vein occlusion (RVO). Radiation therapy, laser therapy and/or surgical resection (removal of the tumor) and/or enucleation are common treatment options for ocular cancer.

In some embodiments, the nanoparticles comprise one type of antisense oligonucleotide contained with the shDNA sequence. In some embodiments, the nanoparticles comprise two types of antisense oligonucleotide contained with the shDNA sequence. In some embodiments, the nanoparticles comprise two or more types of antisense oligonucleotide contained with the shDNA sequence. For example, in some embodiments, the two types of gold-nanoparticles include sequences targeting VEGF mRNA with endoglin mRNA; sequences targeting VEGF mRNA with HIF-1alpha mRNA; and also sequences targeting endoglin mRNA with HIF-1alpha mRNA. In yet other embodiments, the endoglin, VEGF, or HIF-1alpha sequences can be combined with sequences targeting VCAM-1. In some embodiments, each nanoparticle contains about 48-50 of the hairpin DNAs. The ability to suppress multiple mRNAs simultaneously can provide therapeutic response to patients, including those that do not respond to current VEGF therapies or who become refractory to treatment.

In some aspects, disclosed herein is a nanoparticle, comprising:
a short hairpin DNA (shDNA), wherein the shDNA comprises an anti-sense oligonucleotide complementary to a target sequence of an endoglin, HIF-1α, VCAM-1, or VEGF mRNA, or a combination thereof; and
a colloidal gold nanoparticle conjugated to the shDNA.

### EXAMPLES

The following examples are set forth below to illustrate the compounds, nanoparticles, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Gene Silencing and Imaging Nanoparticles

### Nanoparticle Design and Synthesis

Design and Synthesis of hAuNP

DNA oligonucleotides were synthesized that incorporated the anti-sense oligonucleotides disclosed herein (see “Sequences” section) or a scrambled sequence (non-sense sequence). The anti-sense sequence was extensively BLAST-searched to confirm no significant overlap with any other mouse mRNA transcript. The same was performed on the non-sense sequence to confirm non-recognition of any transcribed mouse sequence. The anti-sense oligonucleotide and the nonsense sequences are located within the loop of the hairpin structure. A self-complementary sequence was incorporated into the DNA oligonucleotides, forming the stem of the DNA hairpin. This sequence is largely responsible for the formation and the stability of the hairpin secondary structure. Each DNA-oligonucleotide was computationally designed via energy minimalization to achieve the formation of the hairpin structure. Each of the optimized DNA-oligonucleotide strands was coupled to a fluorescent dye, i.e., an Alexafluor-647 near-infrared (NIR) dye (fluorophore) at the 3′ end. The 5′ end was modified with a thiol group to facilitate linkage to the surface of the gold nanoparticles via an Au—S bond. The hAuNPs were synthesized according to previously described methods (1). Prior to use, thiol-terminated oligonucleotides were subjected to 0.1 M dithiothreitol (DTT) reduction of the 5′ thiol moiety. Excess reducing agent was removed by centrifugal filtration using a filter with a 3K molecular weight cut-off (Amicon Ultracel 3K from Millipore; Billerica, Mass.). The freshly activated 5′ thiol-modified oligonucleotide strands were washed three times with PBS (Life Technologies Corporation; Grand Island, N.Y.) and stored at −80° C. The average diameter of the hAuNP was determined by dynamic light scattering (DLS). The diameters of the gold nanoparticles used to prepare the hAuNP generally ranged from 15-20 nm by transmission electron microscopy (TEM) analysis. The number of DNA-oligonucleotide strands per gold nanoparticle was approximately 48, as quantified by fluorescence measurements after digestion of the hAuNP using DTT, a method described previously (2).

Specificity of Antisense Oligonucleotide (AS) hAuNP

hAuNP were incubated with an exogenous oligonucleotide strand incorporating antisense sequence at concentrations ranging from 3 to 3000 nM. hAuNP were incubated in various media and increased fluorescence activity was observed only in the presence of the oligonucleotide. Rates of hAuNP hybridization with the antisense oligonucleotide depended on the reaction medium; they were slowest in water, increasing in PBS, and fastest in EBM medium. This is consistent with the concept that, media with high ionic concentration can accelerate molecular beacon hybridization kinetics. Though, the double strand stem region is relatively rigid, the probe undergoes spontaneous conformational changes upon hybridization; however, the rate is limited due to an equilibrium between the original beacon conformation. Kinetics of hybridization was faster in EBM, and the fluorescence intensity reaches to maximum within few minutes. Further coupling reaction time had very little effect on the rate or final fluorescence intensity. The slower hybridization reaction in PBS required longer coupling reaction to reach the final fluorescence intensity which was higher than the hybridization-kinetics in EBM, requiring to be monitored for >2 hours.

Stability of AS-VCAM-1 hAuNP

The stabilities of the antisense oligonucleotide hAuNP and citrate capped gold nanoparticles (CT-GNP) were tested and compared in different media. Aggregation of colloidal gold may be detected by changes in absorbance spectra and by TEM. CT-GNP are monodispersed in water; however, as the ionic strength of the aqueous medium is increased, they aggregate as shown in TEM analysis. Changes observed in their absorbance profiles as shown also indicate their aggregation. An absorbance maximum of 520 nm is observed when CT-GNP are monodispersed in water. In contrast, the absorbance profiles in PBS and EBM become broader, which is characteristic of aggregation. Notably, when colloidal dispersions are prepared from antisense oligonucleotide hAuNP in each of these media, there is little change in the absorbance spectrum, indicating a monodispersion.

Specificity of Antisense Oligonucleotide hAuNP for mRNA Target

Samples of total RNA are isolated from mouse retinal microvascular endothelial cells (MRMECs) treated with vehicle or TNF-α for 4 hours and incubated them with antisense oligonucleotide hAuNP or NS hAuNP. A significant fluorescence enhancement is observed in hybridization reactions between antisense oligonucleotide hAuNP and mRNA from TNF-α-induced MRMEC (p<0.05). No signal enhancement is observed in the hybridization reactions with NS hAuNP.

Internalization of hAuNPs by MRMEC

MRMECs were incubated with antisense oligonucleotide or NS hAuNPs, and imaged using TEM. Independent of their nucleotide sequence, hAuNP were observed in the perinuclear region and throughout the cytoplasm in TNF-α and vehicle-treated MRMEC. hAuNP was not observed inside the nucleus of MRMECs. A TEM micrograph demonstrates clusters of NS hAuNP localized in either endosomes or lysosomes throughout the cytoplasm of TNF-α treated MRMECs.

Imaging of VCAM-1 mRNA Expression Levels in Living MRMEC

An increase in antisense oligonucleotide hAuNP-dependent fluorescence enhancement is observed in TNF-α—vs. vehicle-treated MRMECs. The observed cytoplasmic, perinuclear patches of fluorescence are consistent with the localization of hAuNP determined by TEM analysis shown. After prolonged incubation, fluorescence is maintained within the intracellular cytoplasmic compartment, supporting intracellular retention. The NS hAuNP probes are minimally detectable in TNF-α—vs, vehicle-treated MRMECs under the same image acquisition conditions.

Cytotoxicity of hAuNP

Cell viability assays were performed in MRMECs treated with variable concentration of antisense oligonucleotide hAuNPs or NS hAuNPs ranging from 0-5 nM. Calcein AM activation was monitored by fluorescence emission arising from intracellular hydrolysis of the Calcein AM. hAuNPs have no effect on cell viability, indicating that hAuNPs are not acutely toxic to MRMEC. Citrate-capped 15 nm GNPs reduce cell viability, perhaps due to changes in physical properties and aggregation in cell culture medium as shown.

### Example 2. Design and Synthesis of Endoglin Targeted hAuNP

This new type of nanoparticle is designed computationally and conjugated to a hairpin DNA oligonucleotide. The efficacies of these nanoparticles are higher than multiple oligonucleotide-coated gold nanoparticles. In addition, these nanoparticles are synthesized using 1.4 nm spherical gold nanospheres.

Generally, citrate-coated spherical gold nanoparticles are toxic to cells which may be due to formation of aggregates in isotonic solutions (see reference Nanomedicine. 2018 January; 14(1):63-71.doi: 10.1016/j.nano.2017.08.018). However, after the conjugation with hairpin-DNA/RNA, the hAuNP probes exhibited no acute toxicity to the retinal microvascular endothelial cells (MRMECs) as measured by live-dead assay.

### Example 3. Combination Nanoparticles

Two types of gold-nanoparticles were made (15 nm hAuNP with only one target sequence; and 15 nm hAuNP with two target sequences). For example, sequences conjugated to 15 nm gold-nanoparticles include targeting VEGF mRNA with endoglin mRNA; and also sequences targeting VEGF mRNA with HIF-1alpha mRNA. Each nanoparticle contains about 48-50 of the hairpin DNAs.

### REFERENCES

1. Jayagopal, A.; Halfpenny, K. C.; Perez, J. W.; Wright, D. W., Hairpin DNA-Functionalized Gold Colloids for the Imaging of mRNA in Live Cells. Journal of the American Chemical Society 2010, 132 (28), 9789-9796.
2. Taton, T. A., Preparation of gold nanoparticle-DNA conjugates. Curr Protoc Nucleic Acid Chem 2002, Chapter 12, Unit 12 2.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### SEQUENCES

In the following sequences: mG means 2′-MeO protected G, mC means 2′-MeO protected C, mA means 2′-MeO protected A, mT means 2′-MeO protected T, mU means 2′-MeO protected U; 2′-MeO means 2′-O-methyl.

Sequences for DNA-based hAuNP:
MI-2021-mVEGFA V1-5 ALX546:
(SEQ ID NO: 1)
/5ThioMC6-D/TTTTTTTTTT
GCAGCTCTGTCTTTCTTTGGTCTGCGCTGC/3AlexF546/
MI-2021-mVEGFA V1-5:
(SEQ ID NO: 2)
TTTTTTTTTT GCAGCTCTGTCTTTCTTTGGTCTGCGCTGC
MI-2021-mVEGFA V1-5:
(SEQ ID NO: 3)
TCTGTCTTTCTTTGGTCTGC
MI-2021-mVEGF-164 ALX647:
(SEQ ID NO: 4)
/5ThioMC6-D/TTTTTTTTTT
CCGGTAAGGCTCACAGTGATTTTCTACCGG/3AlexF647/
MI-2021-mVEGF-164:
(SEQ ID NO: 5)
TTTTTTTTTT CCGGTAAGGCTCACAGTGATTTTCTACCGG
MI-2021-mVEGF-164:
(SEQ ID NO: 6)
TAAGGCTCACAGTGATTTTCTA
MI_mENG 1:
(SEQ ID NO: 7)
5ThioMC6-
D/TTTTTTTTTTgcagcTGCAACTCAGTTCCATCATTACGGgctgc/3-
Alx488
MI_mENG 1:
(SEQ ID NO: 8)
TTTTTTTTTTgcagcTGCAACTCAGTTCCATCATTACGGgctgc
MI_mENG 1:
(SEQ ID NO: 9)
TGCAACTCAGTTCCATCATTACGG
MI_mENG 1 comp:
(SEQ ID NO: 10)
CCGTAATGATGGAACTGAGTTGCA
MI_mENG 2:
(SEQ ID NO: 11)
5ThioMC6-
D/TTTTTTTTTTgcAGCACTGTGATGTTGACTCTTGGCgctgc/3-
Alx488
MI_mENG 2:
(SEQ ID NO: 12)
TTTTTTTTTTgcAGCACTGTGATGTTGACTCTTGGCgctgc
MI_mENG 2:
(SEQ ID NO: 13)
AGCACTGTGATGTTGACTCTTGGC
MI_mENG 2 comp:
(SEQ ID NO: 14)
GCCAAGAGTCAACATCACAGTGCT
MI_mENG 3:
(SEQ ID NO: 15)
5ThioMC6-
D/TTTTTTTTTTgctcgTTTGACCTTGCTTCCTGGAAAGATcgagc/3-
Alx488
MI_mENG 3:
(SEQ ID NO: 16)
TTTTTTTTTTgctcgTTTGACCTTGCTTCCTGGAAAGATcgagc
MI_mENG 3:
(SEQ ID NO: 17)
TTTGACCTTGCTTCCTGGAAAGAT
MI_mENG 3 comp:
(SEQ ID NO: 18)
ATCTTTCCAGGAAGCAAGGTCAAA
Human sequence:
MI_hENG 1:
(SEQ ID NO: 19)
5ThioMC6-
D/TTTTTTTTTTcgagcGAGAAGTGGACACAGGGACGgctcg/3Alx488
MI_hENG 1:
(SEQ ID NO: 20)
TTTTTTTTTTcgagcGAGAAGTGGACACAGGGACGgctcg
MI_hENG 1:
(SEQ ID NO: 21)
GAGAAGTGGACACAGGGACG
MI_hENG 1 comp:
(SEQ ID NO: 22)
CGTCCCTGTGTCCACTTCTC
MI-mHIF-1a_1:
(SEQ ID NO: 23)
5 ThioMC6-
D/TTTTTTTTTTccggTATTGTCCTTCGTCTCTGTTTTTGAccgg/3-
Alx488
MI_mHIF-1a_1:
(SEQ ID NO: 24)
TTTTTTTTTTccggTATTGTCCTTCGTCTCTGTTTTTGAccgg
MI_mHIF-1a_1:
(SEQ ID NO: 25)
TATTGTCCTTCGTCTCTGTTTTTGA
MI_mHIF-1a_1 comp:
(SEQ ID NO: 26)
TCAAAAACAGAGACGAAGGACAATA
MI-mHIF-1a_2:
(SEQ ID NO: 27)
5ThioMC6-
D/TTTTTTTTTTgcaccGTAAAGAAACATCAGGTAATAggtgc/3-
Alx488
MI-mHIF-1a_2:
(SEQ ID NO: 28)
TTTTTTTTTTgcaccGTAAAGAAACATCAGGTAATAggtgc
MI-mHIF-1a_2:
(SEQ ID NO: 29)
GTAAAGAAACATCAGGTAATA
MI_mHIF-1a_2 comp:
(SEQ ID NO: 30)
TATTACCTGATGTTTCTTTAC
MI-mHIF-1a_3:
(SEQ ID NO: 31)
5ThioMC6-
D/TTTTTTTTTTcgagcATTAAAAGAACATATTAAAAAGAGCgctcg/3-
Alx488
MI-mHIF-1a_3:
(SEQ ID NO: 32)
TTTTTTTTTTcgagcATTAAAAGAACATATTAAAAAGAGCgctcg
MI-mHIF-1a_3:
(SEQ ID NO: 33)
ATTAAAAGAACATATTAAAAAGAGC
MI_mHIF-1a_3 comp:
(SEQ ID NO: 34)
GCTCTTTTTAATATGTTCTTTTAAT
MI_2 Scr:
(SEQ ID NO: 35)
5ThioMC6-
D/TTTTTTTTTTgcagcATAACTCGTCCGTCCGTACCGACCgctgc/3-
Alx488
MI_2 Scr:
(SEQ ID NO: 36)
TTTTTTTTTTgcagcATAACTCGTCCGTCCGTACCGACCgctgc
MI_2 Scr:
(SEQ ID NO: 37)
ATAACTCGTCCGTCCGTACCGACC
MI-2 Scr Comp:
(SEQ ID NO: 38)
GGTCGGTACGGACGGACGAGTTAT
Sequence for RNA-based hAuNP:
MI-01-2022-mHIF-1 2326-RNA seq
(SEQ ID NO: 39)
5′-/5ThioMC6-
D//mCmCmGmGmUmAmUmUmGmUmCmCmUmUmCmGmUmCmUmCmUmGmUm
UmUmUmUmGmAmCmCmGmG/-3′
(SEQ ID NO: 40)
mCmCmGmGmUmAmUmUmGmUmCmCmUmUmCmGmUmCmUmCmUmGmUmUmU
mUmUmGmAmCmCmGmG
(SEQ ID NO: 41)
mUmAmUmUmGmUmCmCmUmUmCmGmUmCmUmCmUmGmUmUmUmUmUmGmA
m
MI-01-2022-mHIF-1 Neg 2326-RNA seq
(SEQ ID NO: 42)
5′-/5ThioMC6-
D//mCmCmGmGmUmUmUmAmGmUmUmCmCmUmGmUmUmCmUmGmUmUmGm
UmCmUmUmCmAmCmCmGmG/-3′
MI-01-2022-mHIF-1 Neg:
(SEQ ID NO: 43)
mCmCmGmGmUmUmUmAmGmUmUmCmCmUmGmUmUmCmUmGmUmUmGmUmC
mUmUmCmAmCmCmGmG
MI-01-2022-mHIF-1 Neg:
(SEQ ID NO: 44)
UmUmUmAmGmUmUmCmCmUmGmUmUmCmUmGmUmUmGmUmCmUmUmCmA
MI-01-2022-mHIF-1 4661-RNA seq
(SEQ ID NO: 45)
5′-/5ThioMC6-
D//mGmCmAmCmCmGmUmAmAmAmGmAmAmAmCmAmUmCmAmGmGmUmAm
AmUmAmGmGmUmGmC /-3′
MI-01-2022-mHIF-1:
(SEQ ID NO: 46)
mGmCmAmCmCmGmUmAmAmAmGmAmAmAmCmAmUmCmAmGmGmUmAmAmU
mAmGmGmUmGmC
MI-01-2022-mHIF-1:
(SEQ ID NO: 47)
mGmUmAmAmAmGmAmAmAmCmAmUmCmAmGmGmUmAmAmUmA
MI-01-2022-mHIF-1 Neg 4661-RNA seq
(SEQ ID NO: 48)
5′-/5ThioMC6-
D//mGmCmAmCmCmAmUmAmGmAmGmAmCmAmAmUmAmUmUmAmGmAmAm
GmAmCmGmGmUmGmC/-3′
MI-01-2022-mHIF-1 Neg:
(SEQ ID NO: 49)
mGmCmAmCmCmAmUmAmGmAmGmAmCmAmAmUmAmUmUmAmGmAmAmGmA
mCmGmGmUmGmC
MI-01-2022-mHIF-1 Neg:
(SEQ ID NO: 50)
mAmUmAmGmAmGmAmCmAmAmUmAmUmUmAmGmAmAmGmAmC
MI-01-2022-mHIF-1 RNA seq-3
(SEQ ID NO: 51)
5′-/5ThioMC6-
D//mCmGmAmGmCmAmUmUmAmAmAmAmGmAmAmCmAmUmAmUmUmAmAm
AmAmAmGmAmGmCmGmCmUmCmG/3′
MI-01-2022-mHIF-1:
(SEQ ID NO: 52)
mCmGmAmGmCmAmUmUmAmAmAmAmGmAmAmCmAmUmAmUmUmAmAmAmA
mAmGmAmGmCmGmCmUmCmG
MI-01-2022-mHIF-1:
(SEQ ID NO: 53)
mAmUmUmAmAmAmAmGmAmAmCmAmUmAmUmUmAmAmAmAmAmGmAmGm
C
MI-01-2022-mENG seq-1-RNA seq
(SEQ ID NO: 54)
/5ThioMC6-D//mGmCmAmGmCmUmGmCmAmAmCmUmCmAmGmUmUmCm
CmAmUmCmAmUmUmAmCmGmGmGmCmUmGmC/-3′
MI-01-2022-mENG seq-1:
(SEQ ID NO: 55)
mGmCmAmGmCmUmGmCmAmAmCmUmCmAmGmUmUmCmCmAmUmCmAmUmU
mAmCmGmGmGmCmUmGmC
MI-01-2022-mENG seq-1:
(SEQ ID NO: 56)
mUmGmCmAmAmCmUmCmAmGmUmUmCmCmAmUmCmAmUmUmAmCmGmG
MI-01-2022-mENG seq-2-RNA seq
(SEQ ID NO: 57)
5′-/5ThioMC6-
D//mGmCmAmGmCmAmCmUmGmUmGmAmUmGmUmUmGmAmCmUmCmUmUm
GmGmCmGmCmUmGmC/-3′
MI-01-2022-mENG seq-2:
(SEQ ID NO: 58)
mGmCmAmGmCmAmCmUmGmUmGmAmUmGmUmUmGmAmCmUmCmUmUmGmG
mCmGmCmUmGmC
MI-01-2022-mENG seq-2:
(SEQ ID NO: 59)
mAmCmUmGmUmGmAmUmGmUmUmGmAmCmUmCmUmUmGmGmC
MI-01-2022-mENG seq-3-RNA seq
(SEQ ID NO: 60)
5′-/5ThioMC6-
D//mGmCmUmCmGmUmUmUmGmAmCmCmUmUmGmCmUmUmCmCmUmGmGm
AmAmAmGmAmUmCmGmAmGmC/-3′.
MI-01-2022-mENG seq-3:
(SEQ ID NO: 61)
mGmCmUmCmGmUmUmUmGmAmCmCmUmUmGmCmUmUmCmCmUmGmGmAmA
mAmGmAmUmCmGmAmGmC
MI-01-2022-mENG seq-3:
(SEQ ID NO: 62)
mUmUmUmGmAmCmCmUmUmGmCmUmUmCmCmUmGmGmAmAmAmGmAmU
Sequence positions in target mRNA:
For ENG seq-1 Mus musculus endoglin Eng),
transcript variant 1, mRNA NM_007932.2:
(SEQ ID NO: 63)
756 GCCAAGAGTCAACATCACAGTGCT 779.
For ENG seq-2 Mus musculus endoglin (Eng),
transcript variant 1, mRNA NM_007932.2:
(SEQ ID NO: 64)
1073 CCGTAATGATGGAACTGAGTTGCA 1096.
For ENG seq-3 Mus musculus endoglin (Eng),
transcript variant 1, mRNA NM_007932.2:
(SEQ ID NO: 65)
1204 ATCTTTCCAGGAAGCAAGGTCAAA 1227.
Mouse VCAM1 antisense oligonucleotide sequence:
(SEQ ID NO: 66)
GCC TCC ACC AGA CTG TAC GAT CCT.

We claim:

1. A nanoparticle, comprising:
a short hairpin DNA (shDNA), wherein the shDNA comprises an anti-sense oligonucleotide complementary to a target sequence of an endoglin mRNA; and
a colloidal gold nanoparticle conjugated to the shDNA.

2. The nanoparticle of claim 1, further comprising a fluorescent dye conjugated to the shDNA.

3. The nanoparticle of claim 1, wherein the shDNA comprises SEQ ID NO:21.

4. The nanoparticle of claim 1, wherein the colloidal gold nanoparticle comprises an additional anti-sense oligonucleotide complementary to a second target sequence.

5. The nanoparticle of claim 1, wherein the additional anti-sense oligonucleotide comprises a sequence complementary to a target sequence of a VCAM-1 or HIF-1α mRNA.

6. The nanoparticle of claim 1, wherein the shDNA comprises about 15-45 nucleotides.

7. The nanoparticle of claim 1, wherein the target sequence of the endoglin mRNA is about 21 nucleotides.

8. The nanoparticle of claim 1, wherein the shDNA comprises at least one chemically modified nucleotide.

9. The nanoparticle of claim 8, wherein the at least one chemically modified nucleotide comprises 2′-O-methyl (2′MeO).

10. The nanoparticle of claim 1, wherein the colloidal gold nanoparticle is about 1.4 nm in diameter.

11. The nanoparticle of claim 1, wherein the nanoparticle is conjugated to the shDNA by a linker.

12. The nanoparticle of claim 1, wherein the linker comprises a C-6 hexane linker.

13. The nanoparticle of claim 2, wherein the fluorescent dye is cyanine-3 (Cy3).

14. A method for inhibiting the expression of an RNA, comprising:
introducing a nanoparticle into a cell or a tissue, the nanoparticle comprising:
a short hairpin DNA sequence (shDNA), wherein the sequence comprises an anti-sense oligonucleotide complementary to a target sequence of an RNA; and
a colloidal gold nanoparticle conjugated to the shDNA;
allowing the anti-sense oligonucleotide to bind the target sequence; and
wherein the binding of the anti-sense oligonucleotide to the target sequence inhibits the expression of the RNA.

15. The method of claim 14, wherein the cell or tissue is an ocular cell or tissue.

16. The method of claim 14, wherein the RNA is selected from an endoglin mRNA, a VCAM-1 mRNA, a HIF-1α mRNA, or a VEGF mRNA.

17. The method of claim 14, wherein the RNA is an endoglin mRNA.

18. The method of claim 14, wherein the RNA is a VCAM-1 mRNA.

19. A method for treating a retinal disease in a subject, comprising:
administering a therapeutically effective amount of a nanoparticle to the subject, wherein the nanoparticle comprises:
a short hairpin DNA sequence (shDNA), wherein the shDNA sequence comprises an anti-sense oligonucleotide complementary to a target sequence of an RNA; and
a colloidal gold nanoparticle conjugated to the shDNA.

20. The method of claim 14, wherein the retinal disease is selected from age-related macular degeneration (AMID), retinopathy of prematurity (ROP), diabetic retinopathy (DR), or branch retinal vein occlusion (BRVO).
